(19) United States
(12) Patent Application Publication
(10) Pub. No.: US 2003/0129578 A1
(43) Pub. Date: Jul. 10, 2003

(54) Method and system for early detection of infectious diseases or symptoms of bioterrorism attacks

(76) Inventor: James R. Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/162,510

(22) Filed: Jun. 4, 2002

Related U.S. Application Data
(60) Provisional application No. 60/344,493, filed on Oct. 26, 2001.

(51) Int. Cl. G06F 17/60; C12Q 1/00; G06F 19/00; G01N 33/48; G01N 33/50

(52) U.S. Cl. 435/4; 702/19; 705/3

(57) ABSTRACT

A method and system for the early detection of infectious diseases, or the symptoms of bioterrorism attacks in a population, includes a plurality of local input devices located with a plurality of individuals geographically dispersed with a population, the local input devices being capable of recording information relating to certain physiological conditions of the respective individuals, and transmitting the information to a central computer via a communication network; and a central computer capable of receiving the transmitted information, of statistically analyzing the information based on a comparison of such information to information transmitted at previous times, to detect patterns of infectious diseases or symptoms of terrorism attacks, and of utilizing the statistical analysis to produce outputs relating to actions to be taken.

### RELATED APPLICATION

The present application is based on Provisional Application No. 60/344,493, filed Oct. 26, 2001, the contents of which are incorporated herein by reference, and the priority date of which application is claimed herein.

### FIELD OF THE INVENTION

The present invention relates to a method, and also a system, for the early detection of infectious diseases, or the symptoms of bioterrorism attacks, in a population.

### BACKGROUND OF THE INVENTION

The difficulties encountered in recognizing the existence of a bioterrorism attack, highlighted by the actual distribution of anthrax spores through the U.S. mails, has established the importance of the early detection of infectious diseases and the need for a system for the immediate reporting of symptoms to a central analysis center to allow patterns of infection to be recognized.

### BRIEF SUMMARY OF THE PRESENT INVENTION

The present invention is broadly directed toward a method and system for reporting the physiological parameters of individuals with suspicious symptoms and the immediate transmission of those parameters to a central location for analysis. More particularly, the invention relates to such a system which engages in dialogs between a central location and a large number of reporting stations in individuals'homes, physicians'offices, hospital emergency rooms and the like. The dialog queries users of the reporting station as to the nature of their symptoms, such as “Do you have a fever, a rash, nausea?” etc. and makes more detailed queries based upon the responses. This dialog is similar to that conducted between a patient and a physician. The inquiries and responses could also relate to the individual's respiratory functions, such as oxygen uptake, vital capacity, tidal volume, and exhalation rate (FEV₁) and to changes in these physiological parameters over time. The transmission of these parameters, or of anomalous parameters potentially indicative of disease, may be made through communications networks to a central location for analysis. The analysis at the central station is performed on a statistical basis so as to detect anomalous physiological measurements arising from confined geographic areas, and thereby the outbreak or spread of infectious diseases, biodisasters, and bioterrorism attacks.

According to one aspect of the present invention, therefore, there is provided a method for the early detection of infectious diseases or the symptoms of bioterrorism attacks in a population, comprising recording by a plurality of individuals geographically dispersed within the population, of information relating to certain physiological conditions of the individuals; transmitting this information to a central computer via a communication network; producing a statistical analysis of the transmitted information, based on the geographic locations of the individuals and the comparison of the transmitted information to information transmitted at previous times, to detect patterns of infectious disease or symptoms of terrorism attacks; and utilizing the statistical analyses to produce outputs relating to action to be taken.

According to further features in the preferred embodiment of the invention described below, the central computer, upon communication therewith being established by any of the individuals, directs a series of inquiries to the respective individuals requesting responses to be transmitted to the central computer. Thus, at least some of the information transmitted to the central computer by the individuals would relate to the presence or absence of symptoms of certain recognized diseases.

Various local input devices may be used to transmit this information to the central computer, including mobile telephones, personal digital assistants (PDAs), personal computers, desktop appliances, interactive TVs, and health condition monitors, such as temperature measuring devices, blood pressure measuring devices, respiratory analyzers, etc.

In the described preferred embodiment, the information is transmitted from the individuals to the central computer via the Internet communication system.

The outputs produced by the central computer, based on the statistical analysis of the inputted information, could include directing the individual to a healthcare agency, such as a hospital or a physician; directing the delivery of selected medications to selected areas; alerting healthcare officials to a particular health condition in a particular geographical location; and/or the issuance of statistical reports based on the statistical analysis.

According to another aspect of the present invention, there is provided a system for the early detection of infectious diseases or the symptoms of bioterrorism attacks on a population, comprising a plurality of local input devices located with a plurality of individuals geographically dispersed with a population, the local input devices being capable of recording information relating to certain physiological conditions of the respective individuals, and transmitting the information to a central computer via a communication network; and a central computer capable of receiving the transmitted information, of statistically analyzing the information based on a comparison of such information to information transmitted at previous times, to detect patterns of infectious diseases or symptoms of terrorism attacks, and of utilizing the statistical analysis to produce outputs relating to actions to be taken.

These local input devices may be operated by the individuals themselves at home or work, or by care providers in the home, the workplace, institutions of learning, physicians'offices, public health clinics, hospitals, emergency rooms, or other appropriate facilities. The measurements may be used to monitor various physiological parameters and detect infectious diseases or suspicious respiratory changes in an individual for the benefit of the individual. Such measurements from large collections of individuals across various geographical, social, occupational, and other groups may thus be collected and transmitted to one or more central stations for appropriate statistical analysis. Group anomalies and trends in those anomalies can thus be identified and analyzed to discover possible outbreak or spread of infectious diseases on a near real-time basis.

Such a system would be advantageous for early detection of the health effects from external causes, such as bioterrorism, introduction of new viruses, environmental events, hazardous chemical contaminations, and other causes.

Further features and advantages of the invention will be apparent from the description below.

### BRIEF DESCRIPTION OF THE DRAWINGS

The invention is herein described, by way of example only, with reference to the accompany drawings, wherein:

FIG. 1 is a general block diagram illustrating one form of system constructed in accordance with the present invention;

FIG. 2 is a block diagram illustrating the construction and operation of each of the local input devices in the system of FIG. 1;

FIG. 3 is a block diagram illustrating the construction and operation of the central computer in the system of FIG. 1; and

FIG. 4 is a flowchart describing a typical operation of the system of FIG. 1.

### DESCRIPTION OF A PREFERRED EMBODIMENT

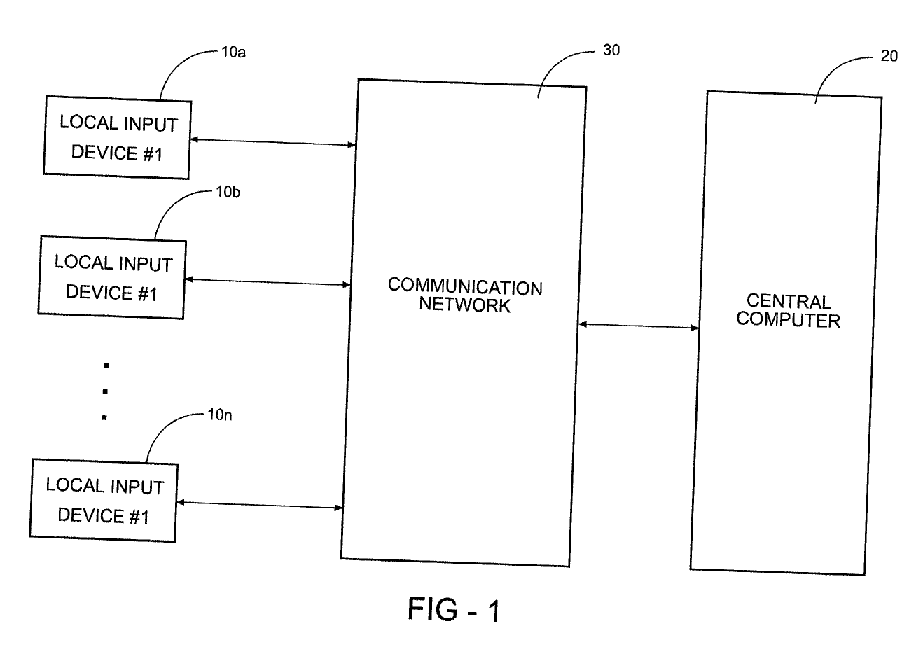

FIG. 1 is a block diagram illustrating the basic components of a system constructed in accordance with the present invention for the early detection of infectious diseases, or the symptoms of bioterrorism attacks in a population.

The system illustrated in FIG. 1 includes a plurality of local input units 10a-10n located with a plurality of individuals geographically dispersed within the population. Each of the local input units 10a-10n is capable of recording information relating to certain physiological conditions of the individuals, and of transmitting this information to a central computer, generally designated 20, via a communication network 30. The central computer 20 is capable of receiving the transmitted information, of statistically analyzing this information based on the comparison of the transmitted information to information transmitted at previous times, and to detect patterns of infectious diseases or symptoms of terrorism attacks. The central computer utilizes this statistical analysis to produce outputs relating to various actions that may be taken as a result of the analysis.

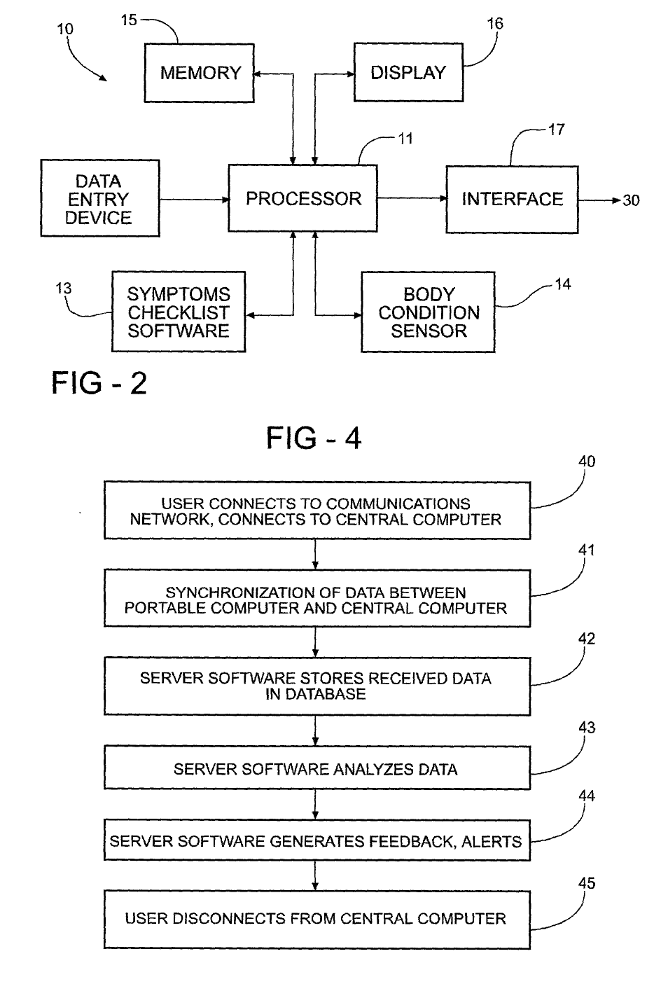

As will be described more particularly below, the system illustrated in FIG. 1, and more particularly illustrated in FIGS. 2-4, enables the early detection of infectious diseases and physiological changes resulting from bioterrorism attacks or other external causes, by the central computer 20 receiving, at a central location, various physiological measurements and symptoms (runny nose, sore throat, etc.) of individuals from widely dispersed geographic areas. Preferably, the measurements constitute a scientific based statistic sampling both in terms of population concentration and geographic dispersion. Statistical techniques like those which have been developed for polling or audience measurement can be used to detect anomalous patterns or trends in specific geographic, occupational, or experiential population groups without the need for physiological measurements from a large number of individuals in any particular population group. For example, harmful chemical exposure to a population in a particular region may be detectable through the analysis and comparison of physiological parameters across an entire state that includes that region by exposing the concentration of relevantly patterned data in that population group. The population group may be defined or represented by any number of variables, including geographical residence or physical location, occupation, attendance at an event or institution, resent visitation to a country or other location, exposure to a health hazard or other variable.

Various physiological parameters such as body temperature, heart rate and the like, as well as subjective symptoms such as the presence of rashes or the feeling of nausea, could be fed into the system from individuals spread over various population groups, and statistically analyzed to detect patterns of measurement or changes in measurement patterns over time which are indicative of infectious disease or physiological parameters deriving from bioterrorism attacks or other health hazards.

When individuals perform their own physiological measurements, the measurements may be fed into a communications network for transmission to a central location by connecting the measurement devices, through wired or wireless connections, to PDAs or cell phones and the like, which could then be connected to the network by docking with personal computers connected to the network or directly through the cellular connections or other suitable communications technologies. Alternatively, the individuals could read the measurements, as well as symptoms, of the physiological sensors and manually enter the measurements into data transmission devices such as personal computers, PDAs, wireless communicators or interactive TV connections to transmit them to the central location.

At central measurement stations such as physicians' offices, public healthcare clinics, supervised care facilities or hospital emergency rooms, the physiological measurements could be fed into the network through personal computers or specialized desktop devices such as the type shown in U.S. Pat. No. 6,101,478, the contents of which are incorporated herein by reference.

FIG. 2 is a block diagram illustrating the main components of the local input devices 10a-10n, or at least some of these devices. Thus, as shown in FIG. 2, the local input device, therein generally designated 10, includes a processor 11 having a data entry device 12, such as a keyboard, touch screen, or other tactile input mechanism, voice recognition or other common data entry mechanism, for entering various physiological parameters of the individual, such as body temperature, heart rate, and the like, as well as subjective symptoms, such as the presence of rashes or the feeling of nausea.

Each local input device 10, or at least some of them, could further include software for gathering information as to particular symptoms. For example, the software could include a checklist of symptoms 13 from which the individual could select the one or ones most appropriate in the individual's case in answer to an inquiry from the central computer 20.

Each data entry device, or some of them, could further include a body condition sensor 14 which senses a body condition of the individual, such as body temperature, heart rate, blood pressure, or the like for transmission to the central computer 20.

Each local input device 10, or some of them, includes a memory 15 for storing data, a display 16 for displaying such data, and an interface 17 for interfacing the local input device with the communication network 30 for transmission to the central computer 20. As indicated earlier, the local input devices 10a-10n could include mobile telephones, PDAs, personal computers, desk appliances, interactive TVs, health condition monitors, etc. According to a preferred embodiment of the invention, one or more of the local input devices is a respiratory analyzer of the type disclosed in U.S. patent application Ser. No. 09/630,398 to detect physiological parameters relating to disease. Such a device comprises a flow tube having a respiratory connector at one end in the form of a mouthpiece or mask. The other end of the flow tube connects to room air. The flow tube includes flow sensors, preferably of the ultrasonic type, to detect instantaneous exhaled flow volume and possibly inhaled flow volume. The device also includes a gas sensor, preferably an oxygen sensor, to detect the instantaneous oxygen partial pressure of the breath flowing through the tube. By integrating the instantaneous flow volume with the instantaneous oxygen partial pressure, the oxygen uptake of the user can be calculated. Inclusion of additional gas sensors for carbon dioxide, nitric oxide, ketones, and other gases can be included to provide quantification of additional physiological parameters. Similarly, other respiratory parameters, such as tidal volume, vital capacity and FEV₁ may be measured using the device. Further details of the construction and operation of such a respiratory analyzer are available from U.S. patent application Ser. No. 09/630,398, the contents of which are incorporated herein by reference.

These physiological parameters, and/or the rate of change of these parameters, can be used to detect infectious diseases. Resting oxygen uptake, or RMR, is known to increase during infection and may precede body temperature increases. Other respiratory factors measured by the device may decrease during respiratory infection.

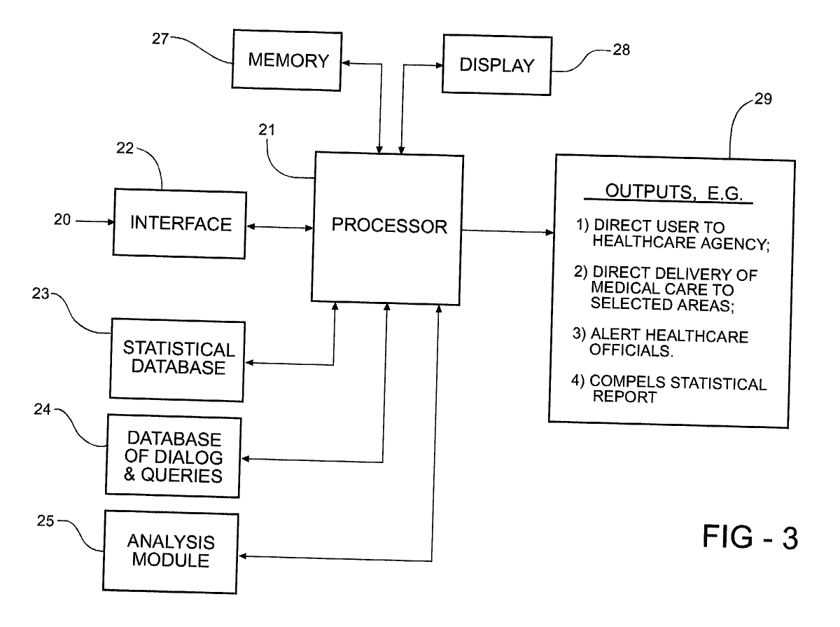

FIG. 3 is a block diagram illustrating the essential components of the central computer 20 at the remote location and communicating with all the local input devices 10a-10n via the communication network 30 to collect the information from those devices which may be relevant to the early detection of infectious diseases, or the symptoms of bioterrorism attacks, in the relevant population. The collection of information by the central computer 20 with the individuals constitutes a dialog with the central computer. Thus, upon the establishment of communication with the central computer by any of the local input devices 10a-10n, the central computer first transmits broad queries as to the status of the patient or reporting individual. For example, the inquiry could be “Do you have a fever?”; or, “Do you feel nauseous?” Based upon the response, the central computer will transmit follow-up questions such as “Have you been in a large group gathering?” or “Have you handled suspicious mail?” The central computer preferably supports a database of dialogs and selects queries from this database, based on responses to previous queries, e.g., in the manner disclosed in U.S. Pat. No. 6,168,563, the contents of which are incorporated herein by reference.

Thus, as shown in FIG. 3, the central computer 20 includes a processor 21 and an interface 22 with a communication network 30. The central computer further includes a database 33 of statistical information relevant to the detection of infectious diseases or symptoms of bioterrorism attacks; and another database 24 of dialogs and queries, which may be selected based on responses to previous queries, or may be in the form of a sequence of queries to elicit certain information from the individuals via their local input devices. Central computer 20 further includes an analysis module 25 for analyzing the information communicated to it by the local input devices, and for performing a statistical analysis of this information, e.g. by comparing the information with the information in the statistical database 23 and/or the database of dialogs and queries 24, to enable an early detection of an infectious disease or a symptom resulting from a bioterrorism attack.

Central computer 20 further includes a memory 26 for storing the data received from the local input devices 10a-10n and the data resulting from its analysis, and a display 28 for displaying this data or selected data as may be desired.

Central computer 20 could be programmed to produce various types of outputs as indicated by block 29 in FIG. 3. Thus, these outputs could include recommendations or directions to the individual directing the individual to a healthcare agency, such as a hospital or physician; directions for the delivery of certain medications to certain geographical areas; or alerts to healthcare officials, alerting them to the possibility of an infectious disease in a particular geographical location, or of a bioterrorism attack in a particular geographical location.

The output of central computer 20 could also include the issuance of statistical reports useful for preparing statistical databases for future use.

As previously indicated, the communication network 30 is preferably the Internet, but it will be appreciated that other public communication systems could be used for establishing communication between the local input devices 10a-10n and the central computer 20.

FIG. 4 is a flowchart illustrating a typical operation of the system of FIGS. 1-3. The flowchart illustrated in FIG. 4 is a variation of the invention described in U.S. patent application Ser. No. 09/721,382, titled “Health Management System with Connection to Remote Computer System” specifically directed toward the early detection of infectious diseases and bioterrorism attacks. That application is incorporated herein by reference. FIG. 4 of the present application is broadly similar to FIG. 4 of Ser. No. 09/721,382.

Thus, as shown in block 40, a user is equipped with a PDA, web enabled cell phone, personal computer, health condition monitor, or other local input device 10a-10n capable of communication with the central computer 20, and running software enabling the input and transmission of symptoms data and physiological measurements to the central computer. The software, as indicated above, incorporates a mechanism for gathering user symptom information, such as a checklist of symptoms from which the user could select, in response to queries by the central computer 20. The queries could be generated by a query based engine that either has a fixed sequence of questions, or relational queries that prompt questions based on the response to prior questions, to identify, or eliminate, specific infectious diseases, or bioterrorism attacks.

The responses by the individuals may be recorded in their respective local input devices 10a-10n by means of touch screens, keyboards or other tactile input mechanisms, voice recognition or other common data entry mechanisms, for entering specific numerical values of physiological parameters such as oxygen uptake, body temperature and the like. This data is then provided to the Internet 30 or other network, through a modem, or ported to a PC which is in turn connected to a modem, a cell phone or the like, as shown by block 41.

At block 42, the central computer 20 receives the data and stores it in its memory.

At block 43 the central computer software analyzes the data. This analysis could include comparison to diagnostic or other databases, and would include analysis of a statistical nature taking into account data from applicable population groups such as the geographic location of the transmissions, the number of transmissions in a geographical area relative to the population of that area, and the correlation of particular parameter measurements or trends with the parameter measurements or trends from other geographic areas and historical measurements or trends captured at other times. This is the type of statistical analysis that the United States Center for Disease Control (CDC) performs, utilizing largely historical information collected by hospitals and the like to detect infectious diseases and their geographic spread.

For example, a statistically significant concentration or high incidence within a specific population group in a short period of time of symptoms such as high fever, headaches, backaches, and bumpy rashes on the face, legs and arms, may be indicative of an outbreak of smallpox in that population group. Spread of the disease could be traced near real-time, as similar symptoms are reported via reporting of the information by collection and transmission device(s) into the central computer stations from different population groups. Such a system could help monitor real time disease migration across population groups and could itself, or in combination with other extrapolation tools, equip healthcare providers with the necessary information to anticipate, predict and prepare for emerging healthcare demands. If a disease is spreading in a certain pattern, for example, preemptive vaccines or supplies could be administered efficiently to optimally reduce its continued spread. In the case of a hazardous materials leak, the migration of symptoms could be predictive of the migration of the hazardous material itself.

At block 44, the software of the central computer 20 generates feedback messages to the originating local input device 10a-10n relative to recommended action that the individuals can take. Preferably, the central computer 20 is also programmed to compile and transmit reports for research groups, public health authorities, healthcare providers, or other applicable entities.

Block 45 relates to the disconnection of the user from the central computer. This operation assumes that the user has been connected during the analysis operation; if not, the operation could be performed while the user is offline and then later transmitted.

It will thus be seen that the invention comprehends: the collection of physiological measurements and symptoms from a collection of population groups; the transmission of this information to a central computer or computers; the statistical analysis and pattern comparison of this information, to detect anomalies and trends indicative of the breakout or spread of infectious diseases, various environmental events having negative or positive health effects, bioterrorism attacks, or other significant causes that impact public health; the transmission of messages from the central station to the individuals or selected population groups, or healthcare agencies; and the compilation and issuance of reports to relevant authorities or care providers.

Thus, medical professionals in physicians'offices, hospitals and the like, as well as individuals at home, may enter information into the system through desktop appliances of the type disclosed in U.S. Pat. No. 6,101,478; from various types of local input devices 10a-10n, which may be connected to the Internet. The data from these devices is transmitted to a secure data center having a central computer 20 via the Internet 30. The software of the central computer selects queries to be directed to the individuals and analyzes their responses to the queries. The response are analyzed both on an individual basis, and on a statistical basis considering their geographic origin and other demographic data such as the age of the respondent, etc. Based upon the responses, additional queries may be sent out to the local input devices 10a-10n, which are more detailed and based upon the responses to the previous queries. The system thus dynamically self-adapts to develop queries based on specific reported conditions.

The results of the statistical analysis may be immediately made available to government agencies such as the CDC and FEMA in order to allow them to detect and respond to infectious diseases or biological threats in the earliest possible time frame.

While the invention has been described with respect to a preferred embodiment thereof, it will be appreciated that this is set forth merely for purposes of example, and that may other variations, modifications and applications of the invention may be made.

What is claimed is:

1. A method for the early detection of infectious diseases or the symptoms of bioterrorism attacks in a population, comprising:
recording by a plurality of individuals geographically dispersed within the population, of information relating to certain physiological conditions of the individuals;
transmitting this information to a central computer via a communication network;
producing a statistical analysis of the transmitted information, based on the geographic locations of the individuals and the comparison of the transmitted information to information transmitted at previous times, to detect patterns of infectious disease or symptoms of terrorism attacks; and
utilizing said statistical analyses to produce outputs relating to action to be taken.

2. The method according to claim 1, wherein said central computer, upon communication therewith being established by any of said individuals, directs a series of inquiries to the respective individuals requesting responses to be transmitted to said central computer.

3. The method according to claim 1, wherein at least some of the information transmitted to the central computer by said individuals relates to the presence or absence of symptoms of certain recognized diseases in the individuals.

4. The method according to claim 1, wherein said information is transmitted to the central computer by at least some individuals via a mobile telephone.

5. The method according to claim 1, wherein said information is transmitted to the central computer by at least some individuals via a personal digital assistant (PDA).

6. The method according to claim 1, wherein said information is transmitted to the central computer by at least some individuals via a personal computer.

7. The method according to claim 1, wherein said information is transmitted to the central computer by at least some individuals via a desktop appliance.

8. The method according to claim 1, wherein said information is transmitted to the central computer by at least some individuals via an interactive TV.

9. The method according to claim 1, wherein said information is transmitted to the central computer by at least some individuals via a health condition monitor.

10. The method of claim 1 wherein at least certain of the physiological conditions are of respiratory parameters to be determined by analysis of an individual's exhalations.

11. The method of claim 10 wherein the respiratory parameters are determined by analyzing the flow rate and constituents of the individual's exhalations through a flow tube.

12. The method according to claim 1, wherein the information is transmitted to the central computer from said individuals via the Internet communication system.

13. The method according to claim 1, wherein said outputs produced by the central computer include directing the individual to a healthcare agency.

14. The method according to claim 1, wherein said outputs produced by the central computer include directing the delivery of selected medications to selected areas.

15. The method according to claim 1, wherein said outputs produced by the central computer include alerting healthcare officials to a particular condition in a particular geographical location.

16. The method according to claim 1, wherein said outputs produced by the central computer include statistical reports based on said statistical analysis.

17. A system for the early detection of infectious diseases or the symptoms of bioterrorism attacks on a population, comprising:
a plurality of local input devices located with a plurality of individuals geographically dispersed with a population, said local input devices being capable of recording informnation relating to certain physiological conditions of the respective individuals, and transmitting said information to a central computer via a communication network; and
a central computer capable of receiving said transmitted information, of statistically analyzing the informnation based on a comparison of such information to information transmitted at previous times, to detect patterns of infectious diseases or symptoms of terrorism attacks, and of utilizing said statistical analysis to produce outputs relating to actions to be taken.

18. The system according to claim 17, wherein said central computer is programmed, upon communication therewith being established by any of said local input units, to direct a series of inquiries to the respective local input units requesting responses therefrom to be transmitted to the central computer, at least some of said inquiries relating to the presence or absence of certain recognized diseases.

19. The system according to claim 17, wherein at least some of said local input devices are mobile telephones.

20. The system according to claim 17, wherein at least some of said local input devices are personal digital assistants (PDAs).

21. The system according to claim 17, wherein at least some of said local input devices are personal computers.

22. The system according to claim 17, wherein at least some of said local input devices are deskctop appliances.

23. The system according to claim 17, wherein at least some of said local input devices are interactive TVs.

24. The system according to claim 17, wherein at least some of said local input devices are healthcare monitors.

25. The system according to claim 17, wherein at least some of said local input devices are respiratory analyzers.

26. The system according to claim 18, wherein said central communication network is the Internet communication network.

27. The system according to claim 17, wherein said outputs produced by the central computer, based on said statistical analysis, include directing the individual to a healthcare agency.

28. The system according to claim 17, wherein said outputs produced by the central computer, based on said statistical analysis, include directing the delivery of selected medications to selected areas.

29. The system according to claim 17, wherein said outputs produced by the central computer, based on said statistical analysis, include alerting healthcare officials to a particular health condition in a particular geographical location.

30. The system according to claim 17, wherein said outputs produced by the central computer, based on said statistical analysis, include the issuance of statistical reports based on said statistical analysis.
